(19) United States
(12) Patent Application Publication
(10) Pub. No.: US 2025/0325593 A1
(43) Pub. Date: Oct. 23, 2025

(54) ENGINEERED MESENCHYMAL STEM CELL AND ITS USE THEREOF

(71) Applicant: National Cheng Kung University, Tainan (TW)

(72) Inventors: Dar-Bin SHIEH, Tainan (TW); Pei-Wen WANG, Tainan (TW); Hsieh-Chih TSAI, New Taipei (TW); Shuian-Yin LIN, Zhubei City (TW)

(21) Appl. No.: 19/083,603

(22) Filed: Mar. 19, 2025

Related U.S. Application Data
(60) Provisional application No. 63/567,482, filed on Mar. 20, 2024.

(51) Int. Cl. A61K 35/28; A61K 9/51; C12N 5/0775

(52) U.S. Cl. CPC A61K 35/28; A61K 9/5115; C12N 5/0668

(57) ABSTRACT

The present invention provides an engineered mesenchymal stem cell (MSC), comprising: a mesenchymal stem cell; and at least one nanoparticle, retained in the MSC, wherein each nanoparticle comprises: a Fe-core and a shell coated on the Fe-core, and the shell has at least one of a hydroxyl group, a carbonyl group and an ether group. Besides, the present invention also provides a method for treating or preventing regenerative disease, comprising: administering an engineered mesenchymal stem cell to a subject in need thereof, wherein the engineered mesenchymal stem cell comprising: a mesenchymal stem cell (MSC); and at least one nanoparticle, retained in the MSC, wherein each nanoparticle comprises: a Fe-core and a shell coated on the Fe-core, and the shell has at least one of a hydroxyl group, a carbonyl group and an ether group.

### CROSS-REFERENCE TO RELATED APPLICATION

This non-provisional application claims the benefit of American Provisional Application No. 63/567,482, filed on Mar. 20, 2024, the contents thereof are incorporated by reference herein.

### FIELD OF THE INVENTION

The present invention relates to a nanoparticle, in particularly to a novel use of nanoparticle in stem cells culture system for regulating the cellular growth and function.

### SEQUENCE LISTING

The Sequence Listing is provided as a file entitled PI-113-060-US-Sequence Listing.xml, created on May 19, 2025, which is 8 kb in size. The information in the electronic format of Sequence Listing is incorporated herein by reference in its entirety.

### BACKGROUND OF THE INVENTION

Mesenchymal stem cells (MSCs) have significant therapeutic potential in regenerative medicine due to their self-renewal capacity and multilineage differentiation potential. Besides, MSCs reside in various in vivo niches and can differentiate into specialized cell types such as adipocytes, osteocytes, chondrocytes, and myocytes.

Comparing to embryonic pluripotent stem cells, MSCs bypass ethical concerns, histocompatibility issues, and the risk of teratoma formation, making them highly attractive for both research and clinical applications. In recently, the demand for MSC culture systems that efficiently support MSC expansion and maintenance is experiencing significant growth, driven by the expanding applications of MSCs in regenerative medicine, disease modeling, and drug development. Specially, the global MSC market was valued at approximately USD 3.12 billion in 2023 and is projected to grow at a compound annual growth rate (CAGR) of 12.92% from 2024 to 2030, reaching an estimated USD 7.27 billion by 2030.

Accordingly, MSCs have ability to expand in ex vivo culture system, coupled with their regenerative potential, have facilitated extensive clinical applications and substantial industrial growth. However, there remains a challenge to optimize MSC culture conditions due to poor adaptability of MSCs to the culture microenvironment, resulting in being unable to massively expand MSCs ex vivo. As above, there is a need to find a method to incubate the mesenchymal stem cells (MSCs) to promote the cellular adaption of MSCs to culture microenvironment.

### SUMMARY OF THE INVENTION

The present invention is made based on the discovery that a nanoparticle has effect on regulating growth and function of mesenchymal stem cell like promoting cellular adaption of MSCs to culture microenvironment, enhancing proliferation and differentiation of the MSCs. It is further discovered that MSCs produced by the method of present disclosure are useful for treating or preventing regenerative disease.

An objective of the present invention is to provide an engineered mesenchymal stem cell (MSC), comprising: a mesenchymal stem cell; and at least one nanoparticle, retained in the MSC, wherein each nanoparticle comprises: a Fe-core and a shell coated on the Fe-core, and the shell has at least one of a hydroxyl group, a carbonyl group and an ether group.

In some embodiments, the shell comprises cellulose, wherein the cellulose comprises one or a mixture of two or more selected from the group consisting of carboxymethyl cellulose (CMC), hydroxypropyl methylcellulose, methylcellulose, hydroxyethyl cellulose and its derivatives thereof. Preferably, the cellulose is CMC.

In some embodiments, a concentration of the at least one nanoparticle in the MSC is at least of 0.1 to 2.5 ppm.

In some embodiments, a concentration of the Fe-core nanoparticles coated with cellulose in the MSC is at least of 0.1 to 2.5 ppm.

In some embodiments, the engineered MSC is manufactured by a method comprising:
a) incubating the MSC with an effective amount of the at least one nanoparticle to obtain the engineered MSCs; wherein the at least one nanoparticle is manufactured by a method, comprising:
   α-1) preparing a nanoparticle forming solution: mixing a solution containing Fe²⁺ and a solution containing cellulose to obtain the nanoparticle forming solution; and
   a-2) adding reducing agent into the nanoparticle forming solution to obtain at least one nanoparticle.

In some embodiments, the solution containing Fe²⁺ has a concentration of 0.1 to 3.0 g/L; and wherein the solution containing cellulose has a concentration of 0.1 to 3.0% (w/w), the cellulose is CMC.

In some embodiments, wherein in the step a-1), preparing a nanoparticle forming solution is performed in the inert gas environment, wherein the inert gas environment is selected from nitrogen gas or hydrogen gas.

In some embodiments, the reducing agent selected from the group consisting of sodium borohydride (NaBH₄), sodium ascorbate and sodium citrate.

In some embodiments, in the step a-2), the reducing agent is added into the nanoparticle forming solution with a ratio, wherein the ratio is in the form of molecular concentration of negative ion in reducing agent ([negative ion in reducing agent]) to molecular concentration of Fe²⁺ ([Fe²⁺]) in the nanoparticle forming solution and is equal to a range of 1 to 10.

Preferably, the reducing agent is NaBH₄; and the reducing agent is added into the nanoparticle forming solution with a ratio, wherein the ratio is in the form of molecular concentration of BH⁴⁻ [BH⁴⁻] in the reducing agent/molecular concentration of Fe²⁺[Fe²⁺] in the nanoparticle forming solution.

In some embodiments, the Fe-core nanoparticles are coated with cellulose shell.

In some embodiments, the surface of Fe-core nanoparticles coated with cellulose contains at least one of a hydroxyl group, a carbonyl group and an ether group.

In some embodiments, the at least one nanoparticle is with a physical diameter of 50 to 85 nm.

In some embodiments, the at least one nanoparticle is with a hydrodynamic size of 58 to 99 nm.

In some embodiments, the at least one nanoparticle has properties include superparamagnetic behavior, negative surface charge and good colloidal stability.

In some embodiments, the effective amount of at least one nanoparticle is of 0.1 to 5.0 μg/mL.

In some embodiments, the method of manufacturing engineered mesenchymal stem cells (MSCs), further comprising:
a-3) washing the at least one nanoparticle with ethanol and vacuum dried, and then keeping the at least one nanoparticle under argon gas until for incubation process.

In some embodiments, wherein after the step a-2), further comprising: collecting the at least one nanoparticle in magnetic field-based environment.

The method of manufacturing engineered MSCs, further comprising: b) differentiation of engineered MSCs: adding a differentiation medium into culture system of the engineered MSCs.

Another objective of the present invention also provides a method for treating or preventing regenerative disease, comprising: administering an engineered mesenchymal stem cell to a subject in need thereof, wherein the engineered mesenchymal stem cell comprising:
a mesenchymal stem cell (MSC); and
at least one nanoparticle, retained in the MSC, wherein each nanoparticle comprises: a Fe-core and a shell coated on the Fe-core, and the shell has at least one of a hydroxyl group, a carbonyl group and an ether group.

In some embodiments, wherein the engineered MSC is administrated to the subject by a route of administration selected from the group consisting of transplantation, local injection and systemic infusion.

In some embodiments, wherein the engineered MSC is administrated to the subject through an administration site selected from the group consisting of an osteoblast-associated site, a chondrocyte-associated site and an adipocyte-associated site.

In some embodiments, wherein the osteoblast-associated site includes: cortical bone, trabecular bone, bone surface, periosteum, bone marrow cavity, osteogenic band or fracture healing site; wherein the chondrocyte-associated site includes: hyaline cartilage, articular cartilage, epiphyseal plate, fibrocartilage, elastic cartilage, cartilage repair site or cartilage of the respiratory tract; and wherein the adipocyte-associated site includes: white adipose tissue, subcutaneous fat, visceral fat, brown adipose tissue, bone marrow fat, fat around organs, mammary gland fat, epicardial fat or perinephric fat.

In some embodiments, in aforementioned method for treating or preventing regenerative disease, a concentration of the at least nanoparticle in MSC is at least of 0.1 to 2.5 ppm.

In some embodiments, in aforementioned method for treating or preventing regenerative disease, a concentration of the Fe-core nanoparticles coated with cellulose in MSC is at least of 0.1 to 2.5 ppm.

### BRIEF DESCRIPTION OF THE DRAWINGS

In the following diagrams, the ZVI@CMC nanoparticles can be named as ZVI@CMC or ZVI@CMC NP alternatively.

FIG. 1 is a diagram of flow chart illustrating a process of incubating MSCs with the ZVI@CMC nanoparticles.

FIG. 2A to 2F are characterization results of ZVI@CMC nanoparticles, wherein FIG. 2A is a TEM picture illustrating the spherical morphology of the ZVI@CMC nanoparticles in scale bar 50 nm; wherein the FIG. 2B is a diagram from XRD measurement illustrating comparing results of CMC curve (with broad peak in diagram, ˜20° to 30° 20; named as CMC herein on the diagram) and ZVI@CMC nanoparticles curve (with lower intensity in diagram; named as ZVI@CMC NP); wherein the FIG. 2C is a diagram from FTIR spectra illustrating functional groups of CMC and ZVI@CMC nanoparticles; wherein the FIG. 2D is a diagram illustrating magnetic hysteresis loop of ZVI@CMC nanoparticles; wherein the FIG. 2E is a diagram illustrating the particle size distribution (mean±SD) of the ZVI@CMC nanoparticles; and wherein the FIG. 2F is a diagram illustrating Zeta potential (mV) measurement results of ZVI@CMC nanoparticles.

FIG. 3A to 3C are biocompatibility results of ZVI@CMC nanoparticles used in MSCs incubation. Wherein FIG. 3A presents cell viability assay showing no significant cytotoxicity; FIG. 3B presents proliferation curve indicating enhancement of MSCs proliferation, particularly at concentrations of 0.5 to 2 μg/mL; and FIG. 3C presents cell cycle analysis. Wherein the ZVI@CMC nanoparticles are used as Experiment group.

FIG. 3D is a diagram illustrating cell proliferation rate (fold) of IONPs (IONP-1, IONP-2 and IONP-3 respectively) used in MSCs incubation. Wherein the IONPs are used as the Comparative group.

FIG. 4A to 4C are a diagram illustrating reverse senescence and mitochondrial DNA stability results upon ZVI@CMC nanoparticles treatment. Wherein FIG. 4A presents the SA-β-gal staining images, and wherein the ZVI@CMC means ZVI@CMC nanoparticles; FIG. 4B presents SA-β-gal activity analyzed quantitatively using flow cytometry at passage 8; FIG. 4C presents analysis of mitochondrial DNA 4977 deletion (fold). **:p<0.001.

FIG. 5A to 5B are a diagram illustrating the cellular uptake and iron accumulation in MSCs treated with ZVI@CMC nanoparticles. Wherein FIG. 5A presents Prussian blue staining associated with iron localization at 24 h and 72 h; FIG. 5B presents the iron quantification in medium and cell pellets after ZVI@CMC nanoparticles treatment for 72 h by inductively coupled plasma mass spectrometry (ICP-MS).

FIG. 6A to 6B are a diagram illustrating expression of MSCs surface markers after ZVI@CMC nanoparticles treatment. Wherein FIG. 6A presents flow cytometry for analysis of MSCs stemness-associated positive markers CD73, CD90, and CD105; FIG. 6B presents relative gene expression analysis of aforementioned MSCs surface markers.

FIG. 7 are staining photographs illustrating effects of ZVI@CMC nanoparticles treatment on MSCs differentiation.

FIG. 8A to 8C are a diagram illustrating gene enrichment analysis of ZVI@CMC nanoparticles treated MSCs compare to nontreated MSCs. Wherein dot plot of FIG. 8A presents pathway enrichment plot; the ridge plot of FIG. 8B presents density distribution of enriched pathways; and the running enrichment score plot of FIG. 8C presents Gene set enrichment analysis (GSEA) enrichment score analysis.

### DETAILED DESCRIPTION OF THE INVENTION

The terms used in this specification are generally within the scope of the present invention and the specific context of each term has its usual meaning in related fields. The specific terms used to describe the present invention in this specification will be described below or elsewhere in this specification, so as to help people in the industry understand the relevant description of the present invention. The same term has the same scope and meaning in the same context. In addition, there is more than one way to express the same thing; therefore, the terms discussed in this article may be replaced by alternative terms and synonyms, and whether a term is specified or discussed in this article does not have any special meaning. This article provides synonyms for certain terms, but the use of one or more synonyms does not mean that other synonyms are excluded.

As used herein, unless the context clearly indicates otherwise, “a” and “the” can also be interpreted as plural. Furthermore, titles and subtitles may be attached to the description for easy reading, but these titles do not affect the scope of the present invention.

By “CMC” it means the carboxymethyl cellulose.

By “MSCs” it means the mesenchymal stem cells.

By “ZVI@CMC nanoparticles” it means the zero-valent iron (ZVI) core nanoparticles were encapsulated within a carboxymethyl cellulose (CMC) shell to form the zero-valent iron (ZVI) nanoparticles coated with CMC. Besides, the ZVI@CMC nanoparticles are also named as ZVI@CMC NP, ZVI@CMC NPs or ZVI@CMC.

By “Fe-core nanoparticles” it means core of nanoparticles is zero-valent Fe-core. Preferably, the “Fe-core nanoparticles coated with CMC” are also named as “ZVI@CMC nanoparticles” or “at least one nanoparticle”.

By “MSCs after treatment” it means the MSCs are obtained from incubating MSCs with an effective amount of Fe-core nanoparticles coated with CMC treatment. Preferably, by “MSCs after treatment” it means the MSCs retaining the Fe-core nanoparticles coated with CMC in the cell through cellular uptake after Fe-core nanoparticles coated with CMC treatment. Preferably, by “MSCs after treatment” it means the modified MSCs/or engineered MSCs after Fe-core nanoparticles coated with CMC treatment.

By “subject” it means the human being or the animals.

In one aspect of the present disclosure, it is discovered that a method for scalable production of mesenchymal stem cells that retain the ZVI@CMC nanoparticles in the MSCs, resulting in promoting cellular adaption to culture microenvironment, enhancing proliferation and differentiation.

It is discovered that a novel engineered mesenchymal stem cell (MSC), comprising: a mesenchymal stem cell; and at least one nanoparticle, retained in the MSC, wherein each nanoparticle comprises: a Fe-core and a shell coated on the Fe-core, and the shell has at least one of a hydroxyl group, a carbonyl group and an ether group.

In some embodiments, the shell comprises cellulose, wherein the cellulose comprises one or a mixture of two or more selected from the group consisting of carboxymethyl cellulose (CMC), hydroxypropyl methylcellulose, methylcellulose, hydroxyethyl cellulose and its derivatives thereof.

In some embodiments, a concentration of the at least one nanoparticle in the MSC is at least of 0.1, 0.2, 0.3, 0.4, 0.5, 1.0, 1.1, 1.2, 1.3, 1.4, 1.5, 1.6, 1.7, 1.8, 1.9, 2.0, 2.1, 2.2, 2.3, 2.4 or 2.5 ppm, but not limited to herein. Preferably, the concentration of the at least one nanoparticle in the MSC is at least of 1.5 ppm. Preferably, by “concentration of the at least one nanoparticle in the MSC” it means intracellular iron concentration.

In some embodiments, a concentration of the Fe-core nanoparticles coated with cellulose in the MSC is at least of 0.1, 0.2, 0.3, 0.4, 0.5, 1.0, 1.1, 1.2, 1.3, 1.4, 1.5, 1.6, 1.7, 1.8, 1.9, 2.0, 2.1, 2.2, 2.3, 2.4 or 2.5 ppm, but not limited to herein. Preferably, the concentration of the Fe-core nanoparticles coated with cellulose in the MSC is at least of 1.5 ppm. Preferably, by “concentration of the Fe-core nanoparticles coated with cellulose in the MSC” it means intracellular iron concentration.

In some embodiments, the engineered MSC is manufactured by a method comprising:
a) incubating the MSC with an effective amount of at least one nanoparticle to obtain the engineered MSCs; wherein the at least one nanoparticle is manufactured by a method comprising:
   a-1) preparing a nanoparticle forming solution: mixing a solution containing Fe²⁺ and a solution containing cellulose to obtain the nanoparticle forming solution; and
   a-2) adding reducing agent into the nanoparticle forming solution to obtain at least one nanoparticle.

In some embodiments, the ddH₂O is used as solvent in preparation of nanoparticle forming solution.

In some embodiments, the solution containing Fe²⁺ has a concentration of 0.1, 0.2, 0.3, 0.4, 0.5, 0.6, 0.7, 0.8, 0.9, 1.0, 1.5, 2.0, 3.0 g/L, but not limited to herein. Preferably, the solution containing Fe²⁺ has a concentration of 0.1 g/L; and wherein the solution containing cellulose has a concentration of 0.1, 0.2, 0.3, 0.4, 0.5, 0.6, 0.7, 0.8, 0.9, 1.0, 1.5, 2.0, 3.0% (w/w), but not limited to herein. Preferably, the solution containing cellulose has a concentration of 0.2% (w/w).

In some embodiments, wherein in the step a-1), preparing a nanoparticle forming solution is performed in inert gas environment, wherein the inert gas environment is selected from nitrogen gas or hydrogen gas. Preferably, the inert gas environment is nitrogen gas environment.

In some embodiments, the reducing agent is selected from the group consisting of sodium borohydride (NaBH₄), sodium ascorbate and sodium citrate. Preferably, the reducing agent is sodium borohydride.

In some embodiments, in the step a-2), the reducing agent is added into the nanoparticle forming solution with a ratio, wherein the ratio is in the form of molecular concentration of negative ion in the reducing agent ([negative ion in reducing agent]) to molecular concentration of Fe²⁺ ([Fe²⁺]) in the nanoparticle forming solution. And wherein, the aforementioned ratio is equal to 1, 2, 3, 4, 5, 6, 7, 8, 9, 10, but not limited to herein. Preferably, the aforementioned ration is equal to 2. Preferably, the reducing agent is sodium borohydride; and wherein the aforementioned ratio is in the form of molecular concentration of BH⁴⁻ [BH⁴⁻] in the reducing agent/molecular concentration of Fe²⁺[Fe²⁺] in the nanoparticle forming solution.

In some embodiments, the zero-valent metal nanoparticles are coated with cellulose shell.

In some embodiments, the surface of the at least one nanoparticle contains at least one of a hydroxyl group, a carbonyl group and an ether group.

In some embodiments, the at least one nanoparticle is with a physical diameter of 50, 51, 52, 53, 54, 55, 56, 57, 58, 59, 60, 65, 70, 71, 72, 73, 74, 75, 76, 77, 78, 79, 80, 81, 82, 83, 84, 85 nm, but not limited to herein. Preferably, the at least one nanoparticle is with a physical diameter of 70.17±14.4 nm.

In some embodiments, the at least one nanoparticle is with a hydrodynamic size of 58, 59, 60, 65, 70, 75, 76, 77, 78, 79, 80, 85, 90, 95, 96, 97, 98, 99 nm. Preferably, the at least one nanoparticle is with a hydrodynamic size of 78.8±19.8 nm.

In some embodiments, the at least one nanoparticle has properties include superparamagnetic behavior, negative surface charge and good colloidal stability.

In some embodiments, in the step of incubating MSCs with an effective amount of the at least one nanoparticle in ex vivo culture system, wherein the effective amount of Fe-core nanoparticles coated with cellulose is concentration of 0.1 μg/mL to 5 μg/mL, including but not limited to, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0 μg/mL. Preferably, the effective amount of the at least one nanoparticle is concentration of 0.5 μg/mL to 2.0 μg/mL.

In some embodiments, the method of manufacturing at least one nanoparticle further comprises: after the step a-2), collecting the at least one nanoparticle in magnetic field-based environment.

In some embodiments, the method of manufacturing at least one nanoparticle further comprises: b) differentiation of engineered MSCs: adding a differentiation medium into culture system of the engineered MSCs.

In some embodiments of the present invention, the Fe-core nanoparticles coated with carboxymethyl cellulose exhibits superior cell proliferation rate in MSCs incubation over that of IONPs.

In some embodiments, Fe-core nanoparticles were encapsulated within a CMC shell to form the Fe-core nanoparticles coated with CMC (also named as at least one nanoparticle), which have the superparamagnetic characteristics. As the Fe-core nanoparticles coated with CMC internalized in MSCs, thereby promoting the MSCs separation efficiency by using high-gradient magnetic columns and purification quality.

In another aspect of the present disclosure, it is discovered that a method for treating or preventing regenerative disease, comprising: administering an engineered mesenchymal stem cell to a subject in need thereof, wherein the engineered mesenchymal stem cell comprising:
a mesenchymal stem cell (MSC); and
at least one nanoparticle, retained in the MSC, wherein each nanoparticle comprises: a Fe-core and a shell coated on the Fe-core, and the shell has at least one of a hydroxyl group, a carbonyl group and an ether group.

In some embodiments, wherein the engineered MSC is administrated to the subject by a route of administration selected from the group consisting of transplantation, local injection and systemic infusion.

In some embodiments, wherein the engineered MSC is administrated to the subject through an administration site selected from the group consisting of an osteoblast-associated site, a chondrocyte-associated site and an adipocyte-associated site.

In some embodiments, wherein the osteoblast-associated site includes: cortical bone, trabecular bone, bone surface, periosteum, bone marrow cavity, osteogenic band or fracture healing site; wherein the chondrocyte-associated site includes: hyaline cartilage, articular cartilage, epiphyseal plate, fibrocartilage, elastic cartilage, cartilage repair site or cartilage of the respiratory tract; and wherein the adipocyte-associated site includes: white adipose tissue, subcutaneous fat, visceral fat, brown adipose tissue, bone marrow fat, fat around organs, mammary gland fat, epicardial fat or perinephric fat.

In some embodiments, after differentiation medium treatment, the engineered MSCs are differentiated to specific tissue composing-associated cells, such as but not limited to, adipocytes, osteocytes, chondrocytes, hepatocytes, cardiocytes, endothelial cells, keratinocytes and myocyte.

In some embodiments, in the engineered MSCs, wherein a concentration of the at least one nanoparticle in MSC is at least of 0.1 to 2.5 ppm.

In some embodiments, in the engineered MSCs, wherein a concentration of the Fe-core nanoparticles coated with cellulose in MSC is at least of 0.1 to 2.5 ppm.

### Examples

The detailed description and preferred embodiments of the invention will be set forth in the following content, and provided for people skilled in the art to understand the characteristics of the invention.

### Material and Methods

### Synthesis of ZVI@CMC Nanoparticles

Mixing a solution containing Fe²⁺ (concentration of 0.1 g/L) and a solution containing carboxymethyl cellulose (CMC) (concentration of 0.2% (w/w)) under nitrogen gas environment for 30 minutes to obtain the nanoparticle forming solution. Then, sodium borohydride of specific ratio was added dropwise into the nanoparticle forming solution and stirred for 10 minutes to obtain a solution containing Fe-core nanoparticles coated with cellulose, wherein the aforementioned ratio is in the form of molecular concentration of BH⁴⁻ ([BH⁴⁻]) in the reducing agent to molecular concentration of Fe²⁺ ([Fe²⁺]) in the nanoparticle forming solution and is equal to 2. And then, placing the solution containing Fe-core nanoparticles coated with cellulose in the magnetic field-based environment, thereby collecting the Fe-core nanoparticles coated with cellulose. Further, washing the collected Fe-core nanoparticles coated with cellulose with ethanol and vacuum dried, and then keep under argon gas until for incubation process.

### Analyze of Superparamagnetic Characteristics of ZVI@CMC Nanoparticles

### 1. Transmission Electron Microscopy (TEM)

Transmission electron microscope (TEM, JEM-1400, JEOL, Japan) was used herein. The measure method is referred to the User Manual.

### 2. X-Ray Diffractometer (XRD) Measurement

XRD patterns of samples were using a SWLS-X-ray Powder Diffraction, BL01C2 beam-line at the NSRRC (National Synchrotron Radiation Research Center, Hsinchu, Taiwan) with a wavelength of 0.77495 Å (16 KeV). The diffraction patterns were recorded using a fixed Mar345 imaging plate detector at a distance of 260 mm from the sample under ambient temperature. The diffracted X-ray will be collected from 2θ=0° to 45°.

### 3. Fourier-Transform Infrared Spectroscopy (FTIR)

FTIR patterns of samples were using FTIR instrument with: Max Resolution: 0.7 cm⁻¹, S/N Ratio: 25,000: 1, Wavenumber Range: 7800 to 350 cm⁻¹.

### 4. Dynamic Light Scattering (DLS)/Zeta Potential Analyzer

The diameter of nanoparticle size and Zeta Potential patterns of samples were using DLS/Zeta Potential Analyzer (NanoPlus Dimensions, Nanoplus-3).

### 5. Magnetic Property Measurement by Scanning Superconducting Quantum Interference Device (SQUID)

The magnetic properties of microcarrier samples were analyzed using a Scanning Superconducting Quantum Interference Device (SQUID; MPMS3, Quantum Design, USA). Approximately 5 mg of microcarrier sample was placed in a non-magnetic sample holder. Magnetization as a function of temperature (M-T) was recorded from 5 K to 300 K under a 100 Oe field in both zero-field-cooled (ZFC) and field-cooled (FC) conditions. Magnetization as a function of applied magnetic field (M-H) was measured at 5 K and 300 K across afield range from −10,000 Oe to +10,000 Oe. All measurements and data analyses were conducted under standardized SQUID operating procedures to ensure precision and reproducibility. Results were further processed and statistically analyzed using Prism software (v9.3.1; GraphPad Software, San Diego, CA, USA) to characterize the magnetic behavior of the ZVI@CMC nanoparticles.

### MSCs Culture and Cell Proliferation Assays

Mesenchymal stem cells (MSCs), isolated from human lipoaspirate tissue and purchased from ThermoFisher (Catalog number: R7788115), were plated in AllPhase xeno-free medium (DuoGenic Stem Cells, Taiwan) at a density of 4,000 cells/cm² and maintained at 37° C. with saturated humidity and 5% CO₂. Upon reaching 80% confluence, MSCs were sub-cultured by treating with Accutase (ThermoFisher) for 3 minutes at 37° C.

In order to further investigate the cell proliferation, the aforementioned cells were then washed, harvested by centrifugation at 1,200 rpm for 5 minutes, and seeded into a 96-well plate at a density of 5,000 cells/well and cultured in 100 μL medium for 24 h. The culture medium was replaced with medium containing different concentrations (0 to 5 μg/mL) of ZVI@CMC nanoparticles for 24, 48, and 72 hours, rinsed with phosphate-buffered saline (PBS, pH 7.4) and incubated in corresponding medium containing 10 μL Cell Counting Kit-8 (CCK-8; Sigma-Aldrich, St. Louis, MO, USA) for each well for 2 h at 37° C. and the absorbance of the solution was measured by a microplate reader at 450 nm wavelength.

### Cell Proliferation Rate and Cell Cycle Analysis

Investigate cytotoxicity and biocompatibility of ZVI@CMC nanoparticles to MSCs.

The group designed in this embodiment

| | Experiment group | Comparative group |
|---|---|---|
| | ZVI@CMC nanoparticles | IONP-1, |
| | | IONP-2, and |
| | | IONP-3, |
| Tested | 0 to 5 μg/mL | 0 to 100 μg/mL |
| Concentration | | |

Wherein, the preparation method of the IONP-1, IONP-2 and IONP-3 in comparative group can be refer to U.S. Pat. No. 10,086,015B2 (nanoparticle preparation in detailed description of preferred embodiments).
Wherein, the IONP-1 is the Glycine IONP (iron oxide nanoparticle); the IONP-2 is the Dimethylglycine IONP; and the IONP-3 is the Trimethylglycine IONP.

To assess MSCs proliferation under ZVI@CMC nanoparticles treatment, a CCK-8 was used to analyze. MSCs were seeded in 96-well plates at a density of 1.5×10⁵ cells/mL, and the CCK-8 reagent was added after incubation at 37° C. for 0, 24, 48, and 72 hours, followed by a 2-hour incubation period. Absorbance at 450 nm was measured to determine cell proliferation. Besides, the proliferation assay performed in Comparative group is basically the same as the aforementioned assay method described in Experiment group.

To assess MSCs cell cycle progression regulation under ZVI@CMC nanoparticles treatment, flow cytometry was performed to analyze. At each time point, 1×10⁶ cells were harvested, fixed in 70% ethanol for overnight, then incubated with 0.5 μg/ml RNase and 50 μg/ml propidium iodide (PI) for 30 min at 37° C. The PI intensity was analyzed by flow cytometry to validate the pharmacological effects of the ZVI@CMC nanoparticles treatment.

### Cell Surface Markers of MSCs

To analyze cell surface markers of MSCs by flow cytometry, MSCs cultured with different concentration of ZVI@CMC nanoparticles were harvested, resuspended in FACS buffer at a concentration of 5×10⁶ cells/mL, and filtered through a 70 μm cell strainer. Using the Human MSC Analysis Kit (BD Biosciences, USA), cells were labeled with an antibody cocktail containing APC anti-CD73, FITC anti-CD90, and PerCP-Cy5.5 anti-CD105. Following a 30 minutes incubation, cells were washed and resuspended in buffer. For quality control, both positive and negative isotype controls were prepared with PE MSC cocktail antibodies. Each isotype and stain control tube contained 100 μL of sample and 2 μL of antibody, incubated for 20 to 30 minutes in the dark. After incubation, 100 μL of PBS was added to each tube before loading onto the BD FACS Canto II (BD Biosciences, USA) flow cytometer. Data analysis was conducted using FlowJo software to assess marker expression levels.

RNA Isolation and Quantitative Real-Time Reverse Transcription Polymerase Chain Reaction (qRT-PCR)

Total mRNA from MSCs cultured on different microcarriers was isolated using Genezol RNA isolation reagent (Geneaid, Taiwan) following the manufacturer's instructions. Real-time RT-PCR was performed using the KAPA SYBR FAST One-Step qRT-PCR Master Mix (Kapa Biosystems, USA) on a StepOnePlus Real-Time PCR system (Applied Biosystems). Gene expression levels were calculated using the ΔΔCt method, normalized to the 18S rRNA gene, and expressed relative to the reference sample.

The following primers shown in Table 1 were used for real-time PCR:

TABLE 1

| Primer | |
|---|---|
| name | sequence |
| CD73 | Forward 5′-CCCATTGACGAACGGAACAA-3′ |
| | (SEQ ID NO: 1); |
| | Reverse 5′-TATACCACGTGAATTCCGCC-3′ |
| | (SEQ ID NO: 2) |
| CD90 | Forward 5′-CGAGAATGCTACCACCTTGC-3′ |
| | (SEQ ID NO: 3); |
| | Reverse 5′-AGCCGGAGTTCACATGTGTA-3′ |
| | (SEQ ID NO: 4); |
| CD105 | Forward 5′-CTCAGGTCCCCAATGCTACC-3′ |
| | (SEQ ID NO: 5); |
| | Reverse 5′-GGTTGAAGGCCAGGTAGAGT-3′ |
| | (SEQ ID NO: 6) |
| 18S | Forward 5′-GCTTAATTTGACTCAACACGGGA-3′ |
| rRNA | (SEQ ID NO: 7); |
| | Reverse 5′-AGCTATCAATCTGTCAATCCTGTC-3′ |
| | (SEQ ID NO: 8) |

For statistical analysis, multiple unpaired t-tests were conducted to compare the mean expression of each target gene across different strains using GraphPad Prism version 9.3.1 (GraphPad Software). A cutoff value of p<0.05 was adjusted for multiple comparisons using the Bonferroni-Dunn method, with p-values below this threshold considered statistically significant.

### MSCs Differentiation Potential

MSCs were seeded on a 12-well plate for differentiation experiments at different densities:
(1) 4,000 cells/cm² for osteogenic differentiation,
(2) 8,000 cells/cm² for adipogenic differentiation, and
(3) 16,000 cells/cm² for chondrogenic differentiation.

After seeding in AllPhase xeno-free medium (DuoGenic, Taiwan), cells were incubated overnight at 37° C. in 5% CO₂ with 95% humidity. After 24 hours, the basal growth medium was replaced with 2 mL of commercial differentiation medium per well for osteogenic (MesenCult™ #05465, Stemcell Technologies, Grenoble, France), adipogenic (MesenCult™ #05413, Stemcell Technologies), or chondrogenic (MesenCult™ #05457, Stemcell Technologies) differentiation. The medium was refreshed twice weekly.

After approximately 14 days of differentiation, cells were fixed with 4% formaldehyde in PBS (pH 7.4) for 30 minutes at room temperature. Differentiation was confirmed with specific staining protocols: Alizarin red S staining solution (Merck Millipore, USA) for osteogenesis, Oil Red O (Sigma-Aldrich, St. Louis, MO, USA) for adipogenesis, and Alcian Blue Solution (Merck Millipore, USA) for chondrogenesis. After staining, cells were washed three times with PBS for 5 minutes each. Samples were then observed and evaluated using an optical microscope (DP72; Olympus, Tokyo, Japan).

### Protein Isolation and Western Blot Analysis

The MSCs were harvested by Accutase (ThermoFisher) for 3 minutes at 37° C. and centrifugation collection of the cell pellet followed by lysis buffer extraction of proteins with protease inhibitors inside. The remaining un-dissolved component was removed by centrifugation at 13000×g at 4° C. The protein concentration was determined by Bio-Rad protein assay (Bio-Rad) using BSA as a standard. Fifty micrograms of total protein were applied for electrophoresis separation in a 10% SDS acrylamide gel along with a molecular weight marker (Fermantas) then electrotransferred to a PVDF membrane in transfer buffer (25 mM Tris-base, 200 mM glycine, and 15% methanol, pH 8.3). The PVDF membrane was blocked with 5% nonfat milk for 1 hours at room temperature, then probed with specific antibodies at 4° C. overnight. After antibodies hybridization, the membrane was washed for 5 times with PBST then incubated with anti-mouse or anti-rabbit immunoglobulin antibodies in 5% non-fat milk or 1 hour followed by visualizing with ECL and detected by a BioSpectrum imaging system.

### Example 1: Incubating MSCs with the ZVI@CMC Nanoparticles

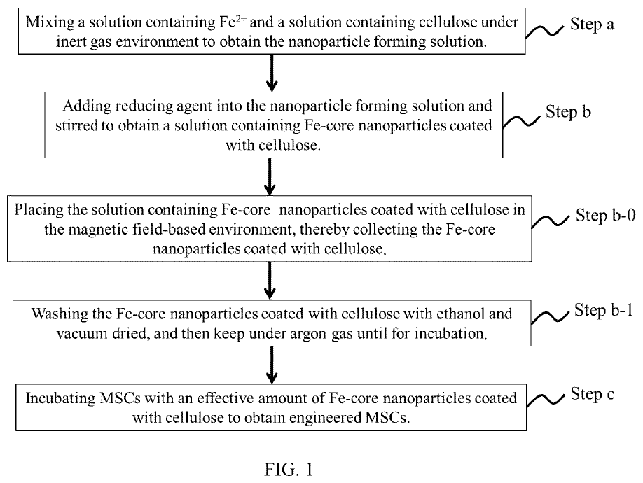

As shown in FIG. 1, the method of manufacturing engineered MSC are as follows:
a) preparing nanoparticle forming solution: mixing a solution containing Fe²⁺ and a solution containing carboxymethyl cellulose (CMC) under nitrogen gas environment for 30 minutes to obtain the nanoparticle forming solution;
b) adding sodium borohydride into the nanoparticle forming solution and stirred for 10 minutes to obtain a solution containing Fe-core nanoparticles coated with CMC;
b-0) collecting nanoparticles: placing the solution containing Fe-core nanoparticles coated with CMC in the magnetic field-based environment, thereby collecting the Fe-core nanoparticles coated with CMC;
b-1) washing the Fe-core nanoparticles coated with CMC with ethanol and vacuum dried, and then keep under argon gas until for incubation process; and
c) incubating MSCs with an effective amount of Fe-core nanoparticles coated with CMC to obtained engineered MSCs.

Wherein the solution containing Fe²⁺ has a concentration of 0.1 g/L; and wherein the solution containing CMC has a concentration of 0.2% (w/w).

Wherein the sodium borohydride (NaBH₄) is added into the nanoparticle forming solution with a ratio, wherein the ratio is in the form of molecular concentration of BH⁴⁻ in reducing agent ([BH⁴⁻]) to molecular concentration of Fe²⁺ ([Fe²⁺]) in nanoparticle forming solution and is equal to 2.

### Example 2: Characterization of ZVI@CMC Nanoparticles

The ZVI core nanoparticles were encapsulated within a CMC shell to form the ZVI@CMC nanoparticles. To confirm the successful synthesis and characterization of ZVI@CMC nanoparticles, multiple analytical techniques were employed.

### 2.1 ZVI@CMC Nanoparticles Having Spherical Morphology

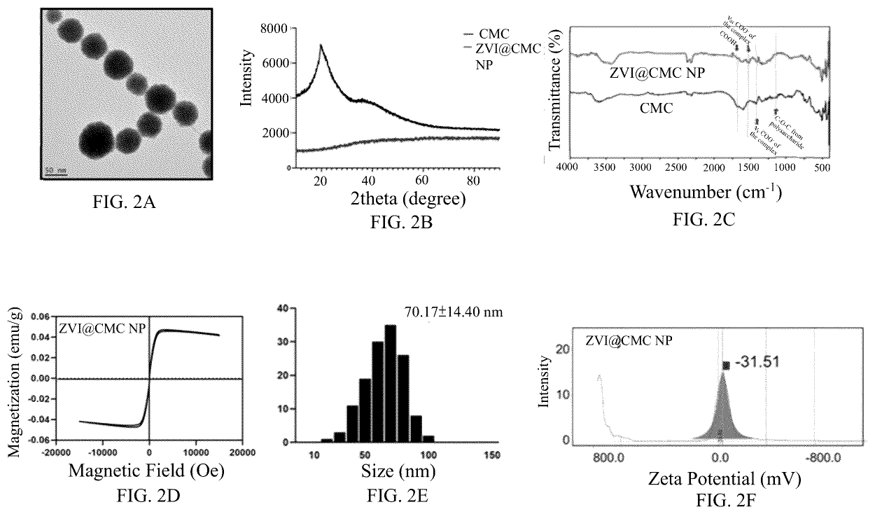

As shown in FIG. 2A, TEM pictures present well-dispersed, spherical morphology of ZVI@CMC nanoparticles with a core-shell structure, presenting ZVI@CMC nanoparticles having successful CMC coating with a mean physical diameter of 70.17±14.4 nm.

### 2.2 ZVI@CMC Nanoparticles Having Disordered or Nanocrystalline Structure

As shown in FIG. 2B, diagram from XRD measurement reveals the CMC curve (with broad peak in diagram, about 200 to 30° 2θ) presents an amorphous structure. Furthermore, the ZVI@CMC nanoparticles curve (with lower intensity in diagram) presents a highly disordered or nanocrystalline structure, presenting stabilization by CMC.

2.3 ZVI@CMC Nanoparticles Having Functionalization with CMC

As shown in FIG. 2C, diagram from the FTIR spectra presents functional groups of CMC and ZVI@CMC nanoparticles. Wherein, surface coating of CMC was achieved through FTIR analysis. FTIR spectra confirm the presence of functional groups such as C═O, C—O—C, and O—H, presenting successful ZVI@CMC nanoparticles functionalization with CMC. Furthermore, aforementioned diagram presents peak shifts between CMC and ZVI@CMC nanoparticles, revealing interactions between the iron core and functional groups of CMC.

### 2.4 ZVI@CMC Nanoparticles Having Superparamagnetic Behavior

As shown in FIG. 2D, the magnetic property measurement presents magnetic hysteresis loop of ZVI@CMC nanoparticles, presenting its superparamagnetic behavior. No significant coercivity or remanence is observed, indicating high magnetization responsiveness.

### 2.5 Particle Size Characters of ZVI@CMC Nanoparticles

As shown in FIG. 2E, the diagram reveals that the ZVI@CMC nanoparticles have the particle size of 70.17±14.4 nm in diameter.

### 2.6 ZVI@CMC Nanoparticles Having Good Stability

As shown in FIG. 2F, the zeta potential of ZVI@CMC nanoparticles is measured at −31.51 mV, presenting strong negative surface charge and good colloidal stability, preventing aggregation.

Besides, results of dynamic light scattering (DLS) measurements illustrates a mean hydrodynamic size of 78.8±19.8 nm of the ZVI@CMC nanoparticles (data not shown).

As shown above, this comprehensive characterization demonstrates that ZVI@CMC nanoparticles possess the required properties for MSCs culture applications, with nanoscale size, functionalization with CMC, superparamagnetic behavior, and good stability. Aforementioned results present potential use of ZVI@CMC nanoparticles in regenerative medicine and stem cell engineering.

### Example 3: Effects of ZVI@CMC Nanoparticles on MSCs Viability, Proliferation, and Cell Cycle Progression

To evaluate the effect of ZVI@CMC nanoparticles on MSCs, cell viability, proliferation, and cell cycle distribution were analyzed.

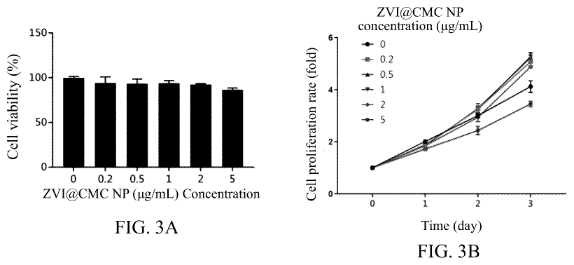

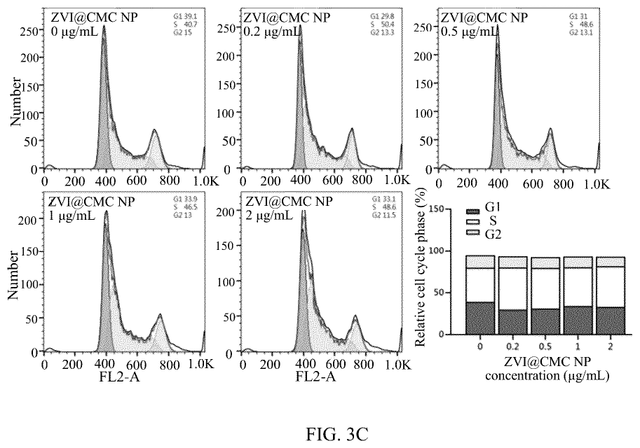

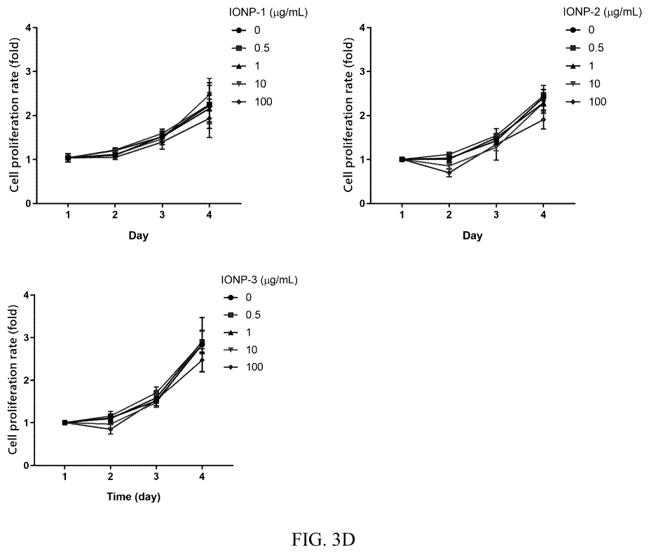

As shown in FIG. 3A, MSCs viability remained above 80-90% across all tested concentrations of ZVI@CMC nanoparticles (0 to 5 μg/mL), suggesting no significant cytotoxicity and presenting the biocompatibility of ZVI@CMC nanoparticles.

As shown in FIG. 3B, cell proliferation analysis presents that ZVI@CMC nanoparticles enhance MSCs proliferation at least 1.7 folds, particularly at concentrations of 0.5 to 2 μg/mL, but with a slight decline at concentration of 5 μg/mL, presenting a threshold effect under ZVI@CMC nanoparticles treatment.

As shown in FIG. 3C, cell cycle progression analysis using flow cytometry reveals an increase in the S-phase population at concentration of 0.5 to 2 μg/mL, indicating that ZVI@CMC nanoparticles promote cell cycle progression and MSCs expansion without inducing arrest.

In contrast, as shown in FIG. 3D, cell proliferation analysis presents that IONPs merely enhance MSCs proliferation rate about 1-fold in the same treated concentration. In particular, as shown in FIGS. 3B and 3D, at the same detection point of Day 3 and the same treated concentration of 0.5 μg/mL, the cell proliferation rate in Experiment group is at least 5 folds, comparatively there is merely 1.2 folds in Comparative group.

Collectively, aforementioned discoveries indicates that ZVI@CMC nanoparticles provide a supportive microenvironment for MSCs culture system by maintaining viability, enhancing proliferation, and promoting cell cycle progression, thereby making them a promising tool for MSCs scalable incubation and be used in regenerative medicine applications.

### Example 4: Effects of ZVI@CMC Nanoparticles on MSCs Senescence and Mitochondrial DNA Integrity

To further investigate the impact of ZVI@CMC nanoparticles on MSCs, cellular senescence and mitochondrial DNA (mtDNA) integrity were assessed.

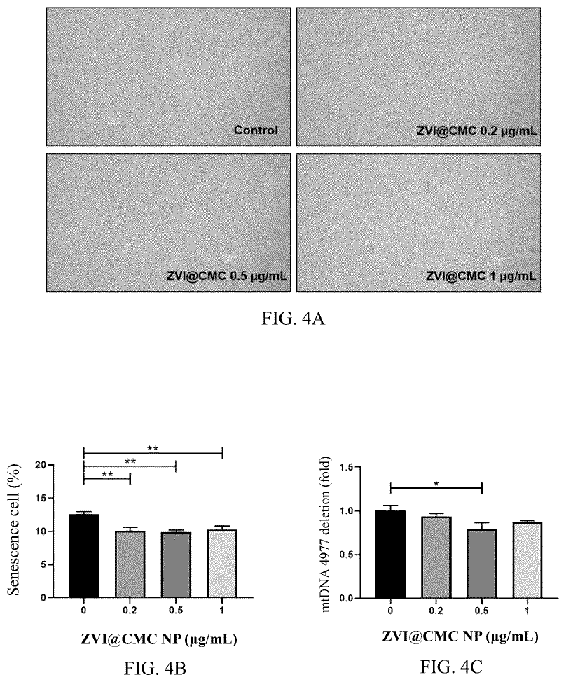

FIG. 4A presents representative staining images of MSCs stained for senescence-associated β-galactosidase (SA-β-gal) activity under different ZVI@CMC nanoparticles concentrations (0, 0.2, 0.5, and 1 μg/mL), presenting a visible reduction in senescent MSCs cells upon ZVI@CMC nanoparticles treatment.

Quantification in FIG. 4B demonstrates a significant decrease in the percentage of senescent cells with increasing ZVI@CMC nanoparticles concentrations, presenting that ZVI@CMC nanoparticles may contribute to delaying MSCs senescence.

Additionally, as shown in FIG. 4C, the mitochondrial DNA integrity was analyzed by measuring mtDNA 4977 deletion levels, revealing a significant reduction in mtDNA deletions in ZVI@CMC nanoparticles-treated MSCs compared to the control.

As above, aforementioned results present that ZVI@CMC nanoparticles not only supports MSCs proliferation but also maintain genomic stability, evaluate the adaption ability to oxidative stress and reverses cellular senescence, thereby reinforcing its potential as an advantageous nanomaterial for stem cells incubation, stem cell engineering and stem cell-based therapies.

### Example 5: Cellular Uptake and Iron Accumulation in MSCs Treated with ZVI@CMC Nanoparticles

To investigate the cellular uptake and accumulation of ZVI@CMC nanoparticles in MSCs, Prussian blue staining was performed to visualize intracellular iron deposition, and using inductively coupled plasma mass spectrometry (ICP-MS, THERMO-ELEMENT XR) to quantify the iron accumulation in MSCs.

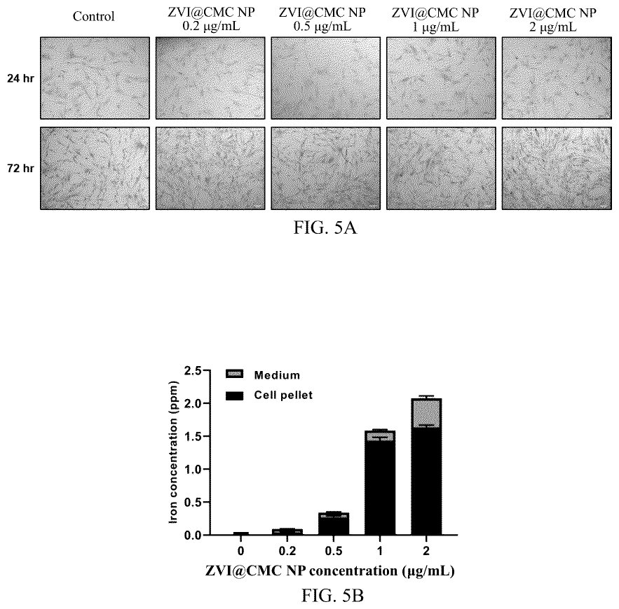

As shown in FIG. 5A, photograph of Prussian blue staining presents the intracellular iron deposition at different time points (24 and 72 hours) and ZVI@CMC nanoparticles concentrations (0, 0.2, 0.5, 1, and 2 μg/mL). There was no significant iron accumulation observed at lower concentrations of ZVI@CMC nanoparticles in MSCs culture system. However, there was increased iron accumulation evidently in MSCs upon treated with higher concentration of ZVI@CMC nanoparticles, particularly at concentration of 1 and 2 μg/mL, presenting the efficient internalization of ZVI@CMC nanoparticles in MSCs.

As shown in FIG. 5B, aforementioned observation was further quantified using ICP-MS, wherein iron concentration measurements in both the culture medium (medium) and MSC pellets (cell pellet) demonstrated that intracellular iron levels increased in a dose-dependent manner after treatment. Notably, most of the iron was retained within the MSCs rather than remaining in the medium, presenting the notion that ZVI@CMC nanoparticles are actively internalized in MSCs. Wherein, an intracellular iron concentration of the ZVI@CMC nanoparticles in the MSC is at least of 1.5 ppm, when comparing experiment group (treatment of 2 μg/mL ZVI@CMC nanoparticles) to control group.

As above, aforementioned findings highlight the efficient cellular uptake, internalization of ZVI@CMC nanoparticles in MSCs and retain of ZVI@CMC nanoparticle in MSCs, thereby reinforcing their use for biomedical applications wherein intracellular iron modulation is beneficial.

### Example 6: Maintenance of MSCs Surface Marker Expression after ZVI@CMC Nanoparticles Treatment

To assess whether ZVI@CMC nanoparticles affect MSCs identity of surface marker expression, the expression of key MSCs surface markers (CD73, CD90, and CD105) were analyzed using flow cytometry and quantitative gene expression analysis.

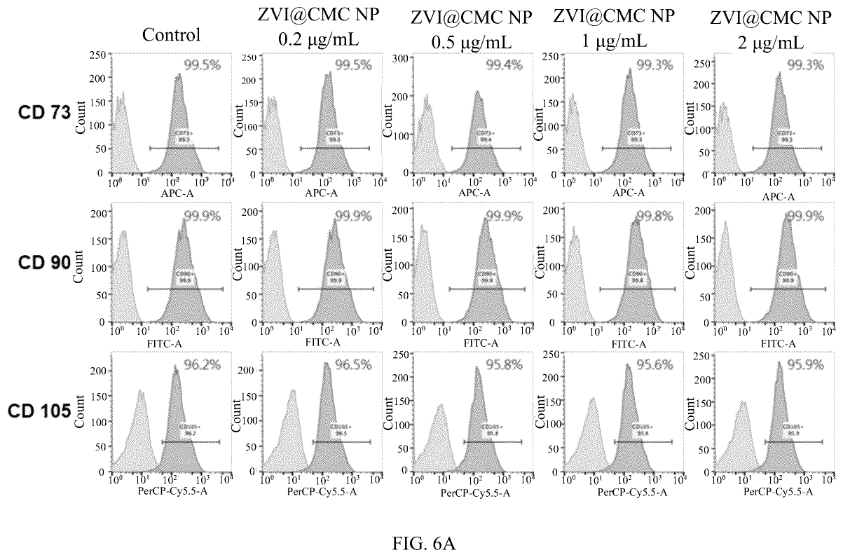

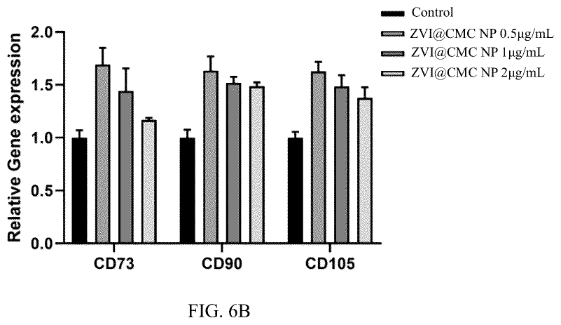

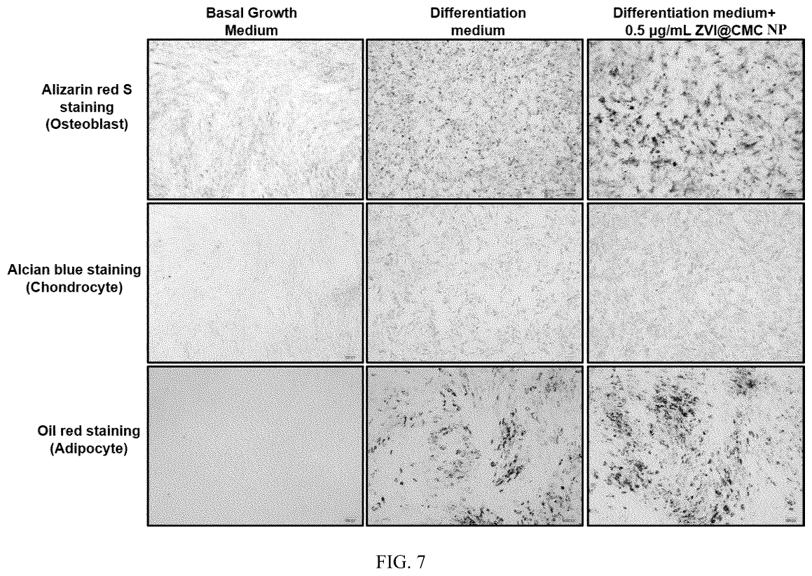

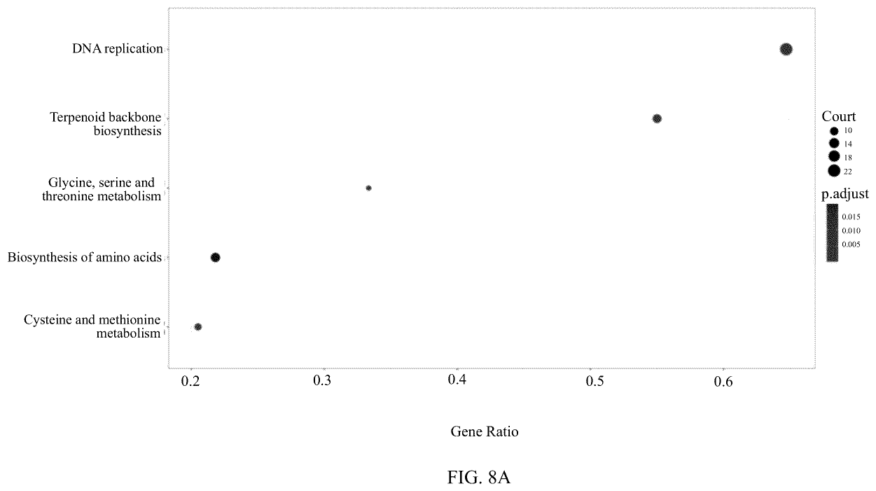

FIG. 6A presents representative flow cytometry histograms for CD73, CD90, and CD105 under different ZVI@CMC nanoparticles concentrations (0, 0.2, 0.5, 1, and 2 μg/mL). The results indicate that MSCs maintain high expression levels of these markers, with up to 95% of the population retaining positive stemness-associated marker expression across all ZVI@CMC nanoparticles treatment conditions and minimal variation compared to control cells.

Further investigation, there is provided by gene expression analysis in FIG. 6B, where relative mRNA expression levels of CD73, CD90, and CD105 remain consistent among different ZVI@CMC nanoparticles treatment groups. These findings suggest that the ZVI@CMC nanoparticles added in MSCs culture medium does not alter the fundamental characteristics of MSCs and supports their phenotypic stability, further presenting its suitability for MSCs culture applications.

### Example 7: Effects of ZVI@CMC Nanoparticles on MSCs Differentiation

As shown in FIG. 7, staining photographs illustrating effects of ZVI@CMC nanoparticles treatment on MSCs differentiation. Compared to the basal growth medium group, there are increase in osteogenic, chondrogenic, and adipogenic differentiation associated staining observed both in differentiation medium group and differentiation medium plus 0.5 g/mL ZVI@CMC nanoparticles group. Furthermore, there are significantly increase in osteogenic, chondrogenic, and adipogenic differentiation associated staining observed in differentiation medium plus 0.5 g/mL ZVI@CMC nanoparticles group compared to differentiation medium group. That is to say, long-term supplementation of 0.5 g/mL ZVI@CMC nanoparticles in MSCs cultures led to enhanced differentiation as evidenced by increased osteoblast, chondrocyte and adipocyte-associated staining.

As above, the aforementioned results confirm promotion osteoblast, chondrocyte, and adipocyte differentiation upon ZVI@CMC nanoparticles addition in MSCs culture system.

### Example 8: Gene Set Enrichment Analysis Reveals ZVI@CMC Nanoparticles Influence on Metabolic and Biosynthetic Pathways in MSCs

To investigate the cellular mechanisms by which ZVI@CMC nanoparticles regulate MSCs in a culture system, the invention performed RNA sequencing (RNA-seq) to analyze gene expression in MSCs cultured with and without ZVI@CMC nanoparticles. Gene set enrichment analysis (GSEA) was used to compare the two conditions, revealing the regulatory effects of ZVI@CMC nanoparticles on cellular processes.

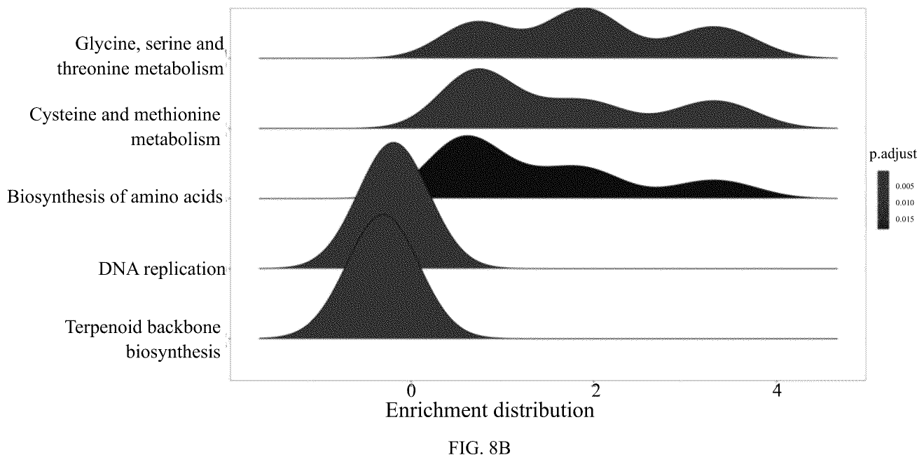

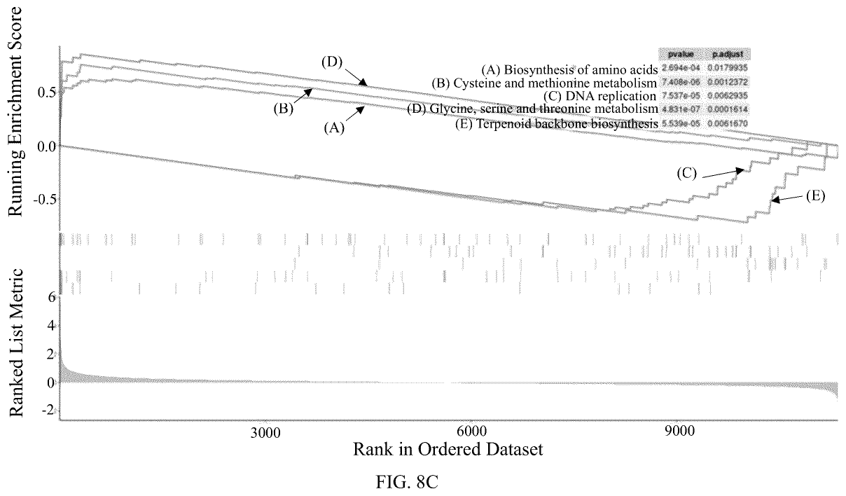

The dot plot as shown in FIG. 8A, it visualizes enriched pathways in ZVI@CMC nanoparticles-treated MSCs compared to untreated controls. The x-axis represents the gene ratio (proportion of pathway-associated genes expressed), while the y-axis lists the different pathways (wherein “DNA replication”, “Terpenoid backbone biosynthesis”, “Glycine, serine and threonine metabolism”, “Biosynthesis of amino acids” and “Cysteine and methionine metabolism”). Wherein dot size corresponds to the number of genes involved, and the color gradient represents the adjusted p-value (q-value), with red indicating higher significance. As shown in the dot plot of FIG. 8A, key pathways enriched in ZVI@CMC nanoparticles-treated MSCs include “DNA replication”, “Terpenoid backbone biosynthesis”, and “Biosynthesis of amino acids”, presenting that ZVI@CMC nanoparticles used in MSCs culture system promote critical metabolic and biosynthetic activities.

As shown in the ridge plot of FIG. 8B, it presents the enrichment score distributions for significantly affected pathways. Distinct peaks for pathways such as “DNA replication” and “Cysteine and methionine metabolism” emphasize their upregulation in ZVI@CMC nanoparticles-treated MSCs. The color gradient reflects adjusted p-values, presenting the statistical significance of these pathways and their potential roles in ZVI@CMC nanoparticles-induced cellular reprogramming in MSCs.

As shown in the running enrichment score plot of FIG. 8C, it dynamically illustrates the GSEA enrichment score analysis results, showing the progression of enrichment scores across the ranked gene dataset. Pathways such as “(A) Biosynthesis of amino acids” and “(C) DNA replication” are significantly enriched in ZVI@CMC nanoparticles-treated MSCs, underscoring ZVI@CMC nanoparticles' ability to enhance pathways related to cell replication, metabolism, and biosynthesis. The legend includes both p-values and adjusted p-values, presenting the relevance and statistical significance of the observed trends.

As above, the aforementioned results collectively demonstrate that ZVI@CMC nanoparticles treatment enhances specific cellular pathways in MSCs, particularly those related to replication, metabolism, and biosynthesis, highlighting ZVI@CMC nanoparticle used in culture system plays potential role in modulating MSCs function.

Above all, the present invention provides ZVI@CMC nanoparticle as a promising tool for MSCs culture applications, thereby presenting a novel approach for optimizing MSCs scalable incubation and maintenance in regenerative medicine and other stem-cell based therapeutic fields.

Specially, the present invention demonstrates that ZVI@CMC nanoparticles provide a beneficial microenvironment for MSCs culture by enhancing cell proliferation while maintaining sternness-associated surface marker expression, and reverse cellular senescence. The uptake and retention of ZVI@CMC nanoparticles within MSCs presents in a dose-dependent manner, without inducing cytotoxic effects, highlighting the biocompatibility of the ZVI@CMC nanoparticles. RNA sequencing and gene set enrichment analysis further revealed that ZVI@CMC nanoparticles treatment influences key metabolic and biosynthetic pathways, including DNA replication, amino acid biosynthesis, and terpenoid backbone biosynthesis. These pathways are crucial for maintaining MSCs function and may contribute to enhanced cellular longevity and differentiation potential. Overall, ZVI@CMC nanoparticles show significant promise as a nanomaterial for MSCs culture applications, with potential implications for regenerative medicine and stem cell-based therapies.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. An engineered mesenchymal stem cell (MSC), comprising:
a mesenchymal stem cell; and
at least one nanoparticle, retained in the MSC, wherein each nanoparticle comprises: a Fe-core and a shell coated on the Fe-core, and the shell has at least one of a hydroxyl group, a carbonyl group and an ether group.

2. The engineered MSC as claimed in claim 1, wherein the shell comprises cellulose, wherein the cellulose comprises one or a mixture of two or more selected from the group consisting of carboxymethyl cellulose (CMC), hydroxypropyl methylcellulose, methylcellulose, hydroxyethyl cellulose and its derivatives thereof.

3. The engineered MSC as claimed in claim 1, wherein a concentration of the at least one nanoparticle in the MSC is at least of 0.1 to 2.5 ppm.

4. The engineered MSC as claimed in claim 1, wherein the engineered MSC is manufactured by a method comprising:
a) incubating the MSC with an effective amount of the at least one nanoparticle to obtain the engineered MSCs; wherein the at least one nanoparticle is manufactured by a method comprising:
a-1) preparing a nanoparticle forming solution: mixing a solution containing Fe²⁺ and a solution containing cellulose to obtain the nanoparticle forming solution; and
a-2) adding reducing agent into the nanoparticle forming solution to obtain the at least one nanoparticle.

5. The engineered MSC as claimed in claim 4, wherein the solution containing Fe²⁺ has a concentration of 0.1 to 3.0 g/L; and wherein the solution containing cellulose has a concentration of 0.1 to 3.0% (w/w), and the cellulose is CMC.

6. The engineered MSC as claimed in claim 4, wherein in the step a-1), preparing a nanoparticle forming solution is performed in inert gas environment, wherein the inert gas environment is selected from nitrogen gas or hydrogen gas.

7. The engineered MSC as claimed in claim 4, wherein the reducing agent is selected from the group consisting of sodium borohydride (NaBH₄), sodium ascorbate and sodium citrate.

8. The engineered MSC as claimed in claim 4, wherein in the step a-2), the reducing agent is added into the nanoparticle forming solution with a ratio, wherein the ratio is in the form of molecular concentration of negative ion in the reducing agent ([negative ion in reducing agent]) to molecular concentration of Fe²⁺ ([Fe²⁺]) in the nanoparticle forming solution and is equal to a range of 1 to 10.

9. The engineered MSC as claimed in claim 8, wherein the reducing agent is NaBH₄; and wherein the ratio is in the form of molecular concentration of BH⁴⁻[BH⁴-] in the reducing agent/molecular concentration of Fe²⁺[Fe²⁺] in the nanoparticle forming solution.

10. The engineered MSC as claimed in claim 1, wherein the at least one nanoparticle is with a physical diameter of 50 to 85 nm.

11. The engineer MSC as claimed in claim 1, wherein the at least one nanoparticle is with a hydrodynamic size of 58 to 99 nm.

12. The engineer MSC as claimed in claim 1, wherein an effective amount of at least one nanoparticle is of 0.1 to 5.0 μg/mL.

13. The engineered MSC as claimed in claim 4, further comprising:
a-3) washing the at least one nanoparticle with ethanol and vacuum dried, and then keeping the at least one nanoparticle under argon gas until for incubation process.

14. The engineered MSC as claimed in claim 4, wherein after the step a-2), further comprising: collecting the at least one nanoparticle in magnetic field-based environment.

15. The engineered MSC as claimed in claim 4, further comprising:
b) differentiation of engineered MSCs: adding a differentiation medium into culture system of the engineered MSCs.

16. A method for treating or preventing regenerative disease, comprising:
administering an engineered mesenchymal stem cell to a subject in need thereof, wherein the engineered mesenchymal stem cell comprising:
a mesenchymal stem cell (MSC); and
at least one nanoparticle, retained in the MSC, wherein each nanoparticle comprises: a Fe-core and a shell coated on the Fe-core, and the shell has at least one of a hydroxyl group, a carbonyl group and an ether group.

17. The method as claimed in claim 16, wherein the engineered MSC is administrated to the subject by a route of administration selected from the group consisting of transplantation, local injection and systemic infusion.

18. The method as claimed in claim 16, wherein the engineered MSC is administrated to the subject through an administration site selected from the group consisting of an osteoblast-associated site, a chondrocyte-associated site and an adipocyte-associated site.

19. The method as claimed in claim 18, wherein the osteoblast-associated site includes: cortical bone, trabecular bone, bone surface, periosteum, bone marrow cavity, osteogenic band or fracture healing site; wherein the chondrocyte-associated site includes: hyaline cartilage, articular cartilage, epiphyseal plate, fibrocartilage, elastic cartilage, cartilage repair site or cartilage of the respiratory tract; and wherein the adipocyte-associated site includes: white adipose tissue, subcutaneous fat, visceral fat, brown adipose tissue, bone marrow fat, fat around organs, mammary gland fat, epicardial fat or perinephric fat.

20. The method as claimed in claim 16, wherein a concentration of the at least one nanoparticle in MSC is at least of 0.1 to 2.5 ppm.


SEQUENCE LISTING (8 sequences)

SEQ ID NO: 1 - DNA, 20 bp, synthetic construct
  source 1..20: mol_type = other DNA
cccattgacgaacggaacaa

SEQ ID NO: 2 - DNA, 20 bp, synthetic construct
  source 1..20: mol_type = other DNA
tataccacgtgaattccgcc

SEQ ID NO: 3 - DNA, 20 bp, synthetic construct
  source 1..20: mol_type = other DNA
cgagaatgctaccaccttgc

SEQ ID NO: 4 - DNA, 20 bp, synthetic construct
  source 1..20: mol_type = other DNA
agccggagttcacatgtgta

SEQ ID NO: 5 - DNA, 20 bp, synthetic construct
  source 1..20: mol_type = other DNA
ctcaggtccccaatgctacc

SEQ ID NO: 6 - DNA, 20 bp, synthetic construct
  source 1..20: mol_type = other DNA
ggttgaaggccaggtagagt

SEQ ID NO: 7 - DNA, 23 bp, synthetic construct
  source 1..23: mol_type = other DNA
gcttaatttgactcaacacggga

SEQ ID NO: 8 - DNA, 24 bp, synthetic construct
  source 1..24: mol_type = other DNA
agctatcaatctgtcaatcctgtc